(19) United States
(12) Patent Application Publication
(10) Pub. No.: US 2017/0325914 A1
(43) Pub. Date: Nov. 16, 2017

(54) ANCHOR IMPLANT

(71) Applicant: Universität Bremen, Bremen (DE)

(72) Inventors: Kamen TUSHTEV, Bremen (DE); Thomas SCHUMACHER, Bremen (DE); Kurosch REZWAN, Bremen (DE)

(21) Appl. No.: 15/520,327

(22) PCT Filed: Oct. 22, 2015
(86) PCT No.: PCT/DE2015/100442

§ 371 Date: Apr. 19, 2017

(30) Foreign Application Priority Data
Oct. 23, 2014 (DE) 10 2014 115 457.6

(51) Int. Cl. A61C 8/00; A61F 2/30

(52) U.S. Cl. CPC A61C 8/0012; A61F 2/30749; A61C 8/0074; A61C 8/0089; A61F 2220/0008

(57) ABSTRACT

The present invention relates to a fixation implant with an implant body which is made substantially of a ceramic material and has an external helical thread with more than one thread web and wherein a thread pitch angle of the external helical thread is in the range from 30° to 90°, and to the use thereof as an alloplastic bone implant, as an alloplastic dental implant, in production engineering, medical engineering, surgery, dental technology and/or implant technology.

The present invention relates to a fixation implant and uses thereof.

There are a number of surgical procedures in which a fixation implant, such as a screw, anchor or nail, is introduced into a patient's tissue and/or bone. In various medical procedures, situations can occur in which bone material from the patient has to be removed in order to secure fixation implants in the bone. One example is the reconstruction of torn cruciate ligaments, since in this case the cruciate ligament graft has to be secured in the bone. In the process, it may be necessary either to replace the ligament with a tendon from the patient's own body or to realign and attach the damaged ligament. The reconstruction of a cruciate ligament in the knee joint is a well-known example of using a tendon graft from the patient's own body.

Different fixation methods for tendon grafts have become commercially established and include both variants close to the joint, such as the use of interference screws, and also variants remote from the joint, such as variants known by the terms “endobutton” or “transfix”. The use in particular of interference screws is at present generally accepted and widespread. A screwdriver is used to introduce these screws along a centrally positioned guide wire into a tunnel drilled into the bone and advanced as far as the graft with constant turning, so that the screw finally engages in the graft. The fixation achieved in this way keeps the graft in its intended place. It is the usual practice in this context to secure the grafts both in the femur and in the tibia.

Conventional interference screws are made of metal, such as titanium or stainless steel, or polymers, e.g. polymers based on lactic acid and/or glycolic acid.

The disadvantage of interference screws based on metal is that they can cause rejection reactions because the material is foreign to the body. In this context, alloy elements such as nickel may sometimes cause auto-immune reactions. Furthermore, interference screws of metal have sharp, self-tapping threads, which frequently damage or sever the graft. These interference screws can also come loose in the course of time. That situation makes it necessary to perform a second operation to remove the screw and correct the damage caused. A further point is that because of the great differences in density between the metal and the bone, artefacts occur in follow-up imaging examinations which make it considerably more difficult to assess the progress of the healing process.

A disadvantage of polymer-based interference screws is the release of acids (lactic acid, for example) during resorption, which can cause the surrounding tissue to be killed off because of the low pH level. This results in a large cavity in the bone, which causes the graft to come loose or fail. An additional problem in securing polymer-based implants is the torsional load when screwing them in and their low strength. In the process, the mechanical forces at work can cause the implant to break or fail. This leads to additional work for the surgeon and a greater burden on the patient.

One example where it is not necessary to use an endogenous tendon graft in order to restore a ligament function is in the shoulder area. Here, wear or overloading cause a ligament injury or torn ligament in the rotator cuff. In this case, the ligament is secured in place and realigned by means of a number of extraction anchors screwed into the bone. These extraction anchors have so far been manufactured from metal or polymer, analogously to the interference screws described above, and similar problems occur to those found with cruciate ligament reconstruction.

EP0625887 B1 discloses an interference screw which is insertable into a tunnel drilled in a bone to secure a graft anchor plug in place at the site of use. The torque acting on the screw during insertion is minimised by providing the screw with a friction-reducing coating.

EP0669110 B1 discloses an orthopaedic interference screw for compression anchoring a bone-tendon graft in a bore formed in a bone mass. That interference screw has a special geometrical structure in order to increase the purchase of the screw and to reduce the fraying or cutting effects caused by the screw on grafts.

DE102008037202 A1 describes an interference screw with an external thread, wherein the threads of the external thread have recesses, at least in some portions.

Finally, DE102010032808 A1 discloses a composition for making an implant, which contains calcium phosphate derivative, binder and compatibiliser. The material described corresponds in its chemical composition almost completely to human bone mineral. It can therefore be integrated into the natural bone and be replaced by the latter a certain time after implantation. A disadvantage of this material, however, is its brittleness and low strength, which have made it difficult to use this material as an implant.

It is now an object of the present invention to provide a fixation implant which overcomes the disadvantages of the state of the art and in particular is made mainly of a ceramic material and possesses satisfactory mechanical properties. In addition, it is intended to be possible to insert the fixation implant into the bone without a great torsional load.

This problem is solved by a fixation implant with an implant body which is made substantially of a ceramic material and has an external helical thread with more than one thread web and wherein a thread pitch angle of the external helical thread is in the range from 30° to 90°.

An external helical thread in the context of the present invention is to be understood as a screw thread with, on its outside, at least two substantially parallel thread webs which are wound about the axis of the implant, extending spirally substantially from a screw head to a screw tip, with a thread pitch angle of 30° to 90°.

According to the invention, it is preferred that the fixation implant is an interference screw or an anchor, such as a suture anchor. An interference screw or a suture anchor are a fixation means which is screwed into a bore that has previously been drilled and is secured in the bore after having been screwed in, by means of an interference fit. Interference screws and suture anchors of this kind are sufficiently well-known in the art.

It is preferred that the ceramic material is a biocompatible ceramic material, preferably a material based on calcium phosphates, calcium silicates, magnesium silicates, zinc silicates and/or strontium silicates.

The term “biocompatible” as used herein refers to a ceramic material which is, for example, biologically active or biologically inert. Materials are described as biocompatible if they have no negative influence on human cell tissue. “Bioactivity” expresses the ability of the ceramic material to enable a direct attachment to the human tissue.

The ceramic material based on calcium phosphates can particularly preferably be selected from the group consisting of hydroxyapatite, tricalcium phosphate, carbonate apatite, fluorapatite and/or combinations thereof.

The ceramic material is preferably a composite material based on polymer and ceramic material or metal and ceramic material.

In a preferred embodiment, the external helical thread has 2, 3, 4, 5 or more thread webs arranged substantially parallel to one another.

The implant body preferably has an axial central channel.

It is likewise preferable that the implant body is configured to be substantially conical or cylindrical.

It is particularly preferable that the external helical thread extends over substantially the entire length of the implant body.

It is particularly preferable that the thread pitch angle of the external helical thread is in the range from at least 45°, preferably 60° to 90°.

It is particularly preferable to insert the fixation implant by exerting pressure, which can be applied without any rotation tool.

The invention also relates to a use of a fixation implant in accordance with the invention as an alloplastic bone implant, as an alloplastic dental implant, in production engineering, medical engineering, surgery, dental technology and/or implant technology.

For the fixation implant of the invention, it has surprisingly been found that first of all, because of the special design of the external helical thread, it can be inserted into a predrilled bone in a self-tapping manner simply by applying axial pressure, and this geometry makes it possible to avoid critical torsional loads. It is not necessary to use a screwdriver, with the torsional loads which that involves. Secondly, this geometry makes it possible to use ceramic materials which were ruled out for the intended medical application because of the torsional moments arising with conventional interference screws. In other words, the shape of the fixation implant enables the use of ceramic material which readily withstands the pressure loads needed for insertion. The fixation implant can be secured to the bone in accordance with the invention in a manner comparable to a wedge, via the contact surface of the external helical thread of the fixation implant. The fixation implant of the invention thus makes it possible to develop a new class of materials for implants of this kind, so that the disadvantageous properties when other classes of materials are used, such as polymers or metals, are avoided. The operative effort in inserting the fixation implant of the invention is minor, and the risk of failure of components as a result of excessive torsional loads (torque) is likewise minimised.

In principle, there are two types of stress, compressive stresses and tensile stresses. The ceramic material used in accordance with the invention reacts very sensitively to tensile stresses, whereas compressive stresses are tolerated to a considerably greater extent.

It is a well-known fact that prior art interference screws are inserted into the drilled tunnel with a screwdriver. This gives rise to very high tensile stresses, which bioactive ceramics, for example, such as calcium phosphates or calcium silicates, cannot withstand, so that they break. With the fixation implant of the invention, with its special screw geometry, no screwdriver is needed for insertion any longer, but only an axial compressive force, such as a hammer for example. For this reason, hardly any tensile stresses arise with the fixation implant of the invention, but primarily compressive stresses, which can be tolerated by the ceramic material. In accordance with the invention therefore, only an axial compressive force is required for inserting the fixation implant, in order to insert the implants. The very design means that the fixation implant “screws itself in”.

Further features and advantages of the fixation implant of the invention will become clear from the following detailed description and the enclosed drawing, in which the Figure shows a fixation implant of the invention, inserted into a bone tunnel together with a ligament, in a section view. FIG. 2 illustrates what is meant by a thread pitch angle with reference to an exemplary screw body.

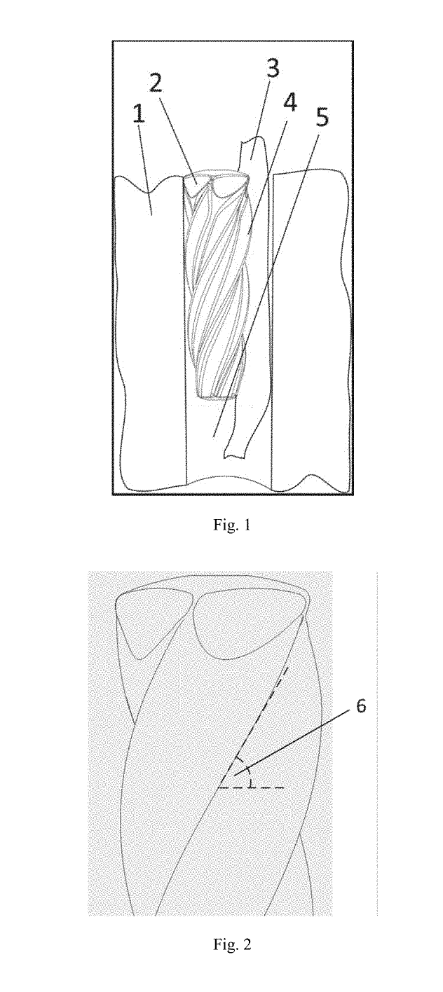

As can be seen from the section view shown in FIG. 1, a fixation implant in the form of an interference screw 2 is shown there in a tunnel 5 inside a bone material 1, together with a tendon or ligament 3. The interference screw 2 has a helical thread 4, the helical thread 4 extending over the entire implant body of the interference screw 2.

In order to insert the interference screw, an orthopaedic surgeon drills a tunnel 5 into the bone material 1, which may, for example, be carried out athroscopically. After the tunnel 5 has been produced, one end of the ligament 3 is positioned as illustrated. The interference screw 2 is then screwed into the tunnel 5 in a self-tapping manner by applying an axial compressive load, in order to secure the ligament 3 in the tunnel. The axial compressive load can be applied in a simple manner by applying a suitable force to the end face of the interference screw 2.

FIG. 2 shows a screw body which clearly illustrates what is meant by a thread pitch angle. According to the invention, it is necessary for the thread pitch angle to be greater than 30°. This kind of pitch angle of the helical thread makes it possible to insert or introduce a fixation implant of the invention without any need to screw it in actively.

The features of the invention disclosed in the above description, the claims and the drawing can be essential to implementing the invention in its various embodiments both individually and in any combination.

1. A fixation implant with an implant body, which is made substantially of ceramic material and has an external helical thread with more than one thread web and wherein a thread pitch angle of the external helical thread is in the range from 30° to 90°.

2. The fixation implant as claimed in claim 1, wherein the fixation implant is an interference screw or an anchor, such as a suture anchor.

3. The fixation implant as claimed in claim 1, wherein the ceramic material is a biocompatible ceramic material, preferably a material based on calcium phosphates, calcium silicates, magnesium silicates, zinc silicates and/or strontium silicates.

4. The fixation implant as claimed in claim 3, wherein the ceramic material based on calcium phosphates is selected from the group consisting of hydroxyapatite, tricalcium phosphate, carbonate apatite, fluorapatite and/or combinations thereof.

5. The fixation implant as claimed in claim 1, wherein the ceramic material is a composite material based on polymer and ceramic material or metal and ceramic material.

6. The fixation implant as claimed in claim 1, wherein the external helical thread has 2, 3, 4, 5 or more thread webs arranged substantially parallel to one another.

7. The fixation implant as claimed in claim 1, wherein the implant body has an axial central channel.

8. The fixation implant as claimed in claim 1, wherein the implant body is configured to be substantially conical or cylindrical.

9. The fixation implant as claimed in claim 1, wherein the external helical thread extends over substantially the entire length of the implant body.

10. Use of a fixation implant as claimed in claim 1 as an alloplastic bone implant, as an alloplastic dental implant, in production engineering, medical engineering, surgery, dental technology and/or implant technology.
